(19) United States
(12) Patent Application Publication
(10) Pub. No.: US 2018/0214197 A1
(43) Pub. Date: Aug. 2, 2018

(54) CIRCULATION SMOKE DISCHARGING SYSTEM

(71) Applicant: OLYMPUS CORPORATION, Tokyo (JP)

(72) Inventors: Kunitoshi HIRAGA, Tama-shi (JP); Nobuyuki FURUKAWA, Hachioji-shi (JP); Satoshi HOMMA, Hachioji-shi (JP); Susumu AONO, Hachioji-shi (JP); Takanori USHIJIMA, Tama-shi (JP)

(73) Assignee: OLYMPUS CORPORATION, Tokyo (JP)

(21) Appl. No.: 15/938,250

(22) Filed: Mar. 28, 2018

Related U.S. Application Data
(63) Continuation of application No. PCT/JP2016/077466, filed on Sep. 16, 2016.

(30) Foreign Application Priority Data
Oct. 1, 2015 (JP) 2015-196107

(51) Int. Cl. A61B 18/14

(52) U.S. Cl. CPC A61B 18/1402; A61B 2218/008; A61B 1/015; A61B 2018/1412; A61M 2202/0225

(57) ABSTRACT

A circulation smoke discharging system includes: a circulation smoke discharging apparatus configured to receive setting output information of a treatment instrument apparatus including a treatment instrument, feed predetermined gas, and perform circulation smoke discharging by sucking smoke generated in a body cavity and gas in the body cavity; a pump for the circulation smoke discharging; an input voltage determination unit configured to determine a pump input voltage corresponding to a circulation flow rate of the circulation smoke discharging in accordance with the setting output information of the treatment instrument apparatus; and a circulation flow rate control unit configured to control the circulation flow rate by outputting the pump input voltage to the pump.

This application is a continuation application of PCT/JP2016/077466 filed on Sep. 16, 2016 and claims benefit of Japanese Application No. 2015-196107 filed in Japan on Oct. 1, 2015, the entire contents of which are incorporated herein by this reference.

### BACKGROUND OF THE INVENTION

### 1. Field of the Invention

The present invention relates to a circulation smoke discharging system.

### 2. Description of the Related Art

In a conventional electric surgical apparatus as disclosed in, for example, Japanese Patent Application Laid-Open Publication No. 2006-288553, a smoke discharging apparatus feeds carbon dioxide into an abdominal cavity to externally discharge smoke generated in treatment with an electrocautery scalpel in accordance with an output value of the electrocautery scalpel.

### SUMMARY OF THE INVENTION

A circulation smoke discharging system according to an aspect of the present invention includes: a reception unit configured to receive setting output information of a treatment instrument apparatus including a treatment instrument; a circulation flow rate information setting unit storing a plurality of pieces of the setting output information and a plurality of circulation flow rates associated with the plurality of pieces of the setting output information, and configured to set one of the plurality of circulation flow rates in accordance with the setting output information; a circulation smoke discharging apparatus configured to feed gas at the circulation flow rate set by the circulation flow rate information setting unit in accordance with a result of the reception by the reception unit and perform circulation smoke discharging by sucking smoke generated in a body cavity and gas in the body cavity; a pump for the circulation smoke discharging; an input voltage determination unit configured to determine a pump input voltage corresponding to the circulation flow rate; and a circulation flow rate control unit configured to control the circulation flow rate by outputting the pump input voltage to the pump.

### BRIEF DESCRIPTION OF THE DRAWINGS

FIG. 1 is a block diagram illustrating a configuration of a circulation smoke discharging system according to an embodiment of the present invention;

FIG. 2 is a block diagram illustrating a configuration of a circulation smoke discharging apparatus of the circulation smoke discharging system according to the embodiment of the present invention;

FIG. 3 is a flowchart illustrating a process of pneumoperitoneum processing by the circulation smoke discharging system according to the embodiment of the present invention;

FIG. 4 is a flowchart illustrating a process of circulation smoke discharging processing by the circulation smoke discharging apparatus of the circulation smoke discharging system according to the embodiment of the present invention;

FIG. 5 is an explanatory diagram for description of an exemplary flow rate voltage table for the circulation smoke discharging system according to the embodiment of the present invention;

FIG. 6 is a flowchart illustrating a process of relief processing by the circulation smoke discharging system according to the embodiment of the present invention; and

FIG. 7 is an explanatory cross-sectional view for description of a configuration of a distal end of an endoscope insertion section.

### DETAILED DESCRIPTION OF THE PREFERRED EMBODIMENTS

An embodiment of the present invention will be described below with reference to the accompanying drawings.

### (Configuration)

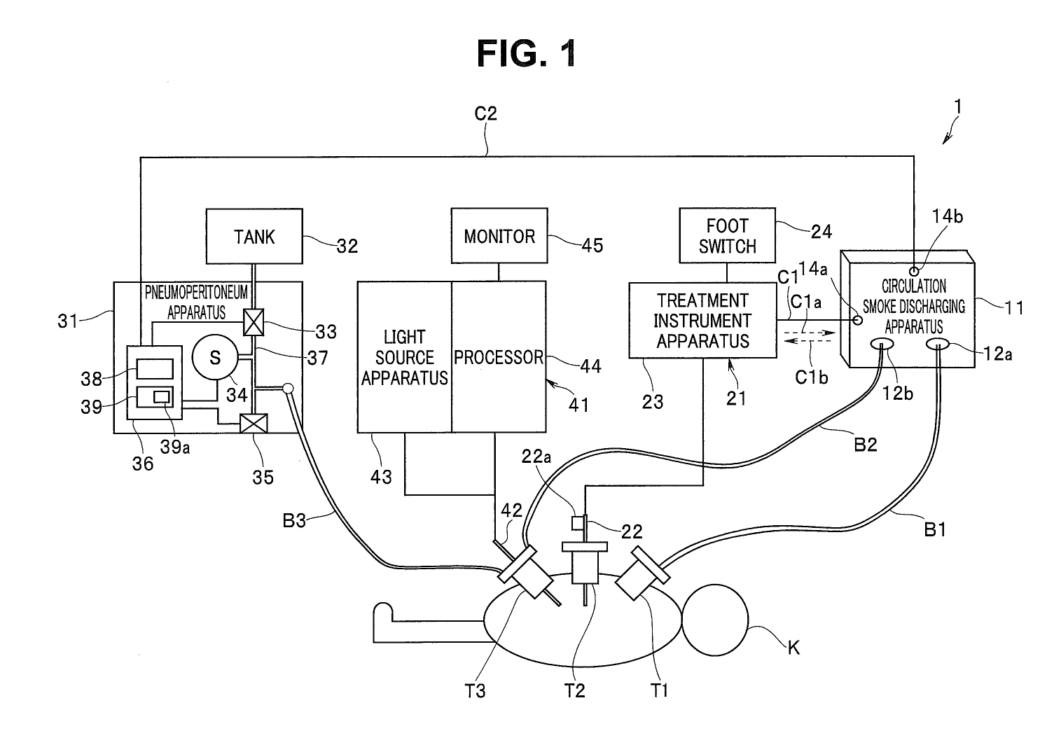

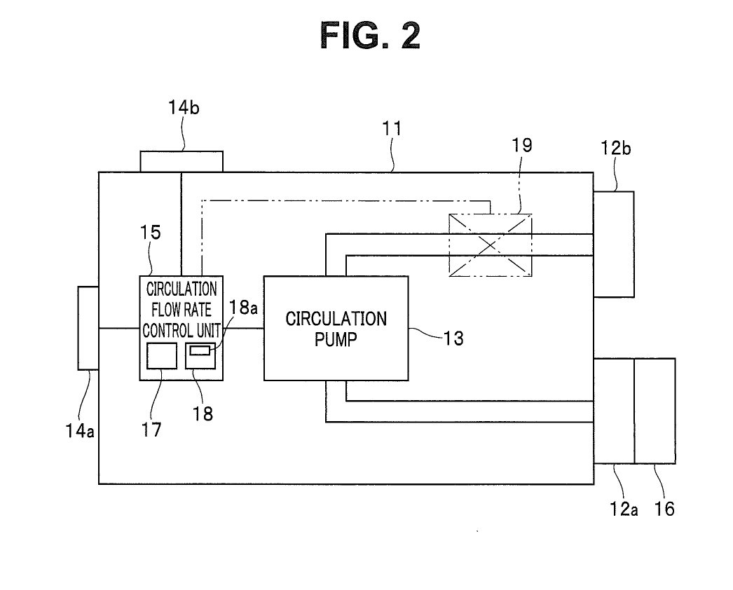

FIG. 1 is a block diagram illustrating a configuration of a circulation smoke discharging system 1 according to an embodiment of the present invention. FIG. 2 is a block diagram illustrating a configuration of a circulation smoke discharging apparatus 11 of the circulation smoke discharging system 1 according to the embodiment of the present invention.

The circulation smoke discharging system 1 according to the embodiment is used in surgery under endoscope observation in which gas such as carbon dioxide is fed to expand inside of a body cavity of a patient K and treatment is performed with a treatment instrument such as an electrocautery scalpel 22.

As illustrated in FIG. 1, the circulation smoke discharging system 1 includes the circulation smoke discharging apparatus 11, a treatment instrument apparatus 21, a pneumoperitoneum apparatus 31, and an endoscope apparatus 41. A suction trocar T1, a treatment instrument trocar T2, and an air feeding trocar T3 are inserted into the body cavity of the patient K.

As illustrated in FIG. 2, the circulation smoke discharging apparatus 11 includes a suction port 12a, an air feeding port 12b, a circulation pump 13, connection units 14a and 14b, and a circulation flow rate control unit 15. The circulation smoke discharging apparatus 11 is configured to receive setting output information of a treatment instrument apparatus including a treatment instrument, feed predetermined gas, and perform circulation smoke discharging by sucking smoke generated in the body cavity and gas in the body cavity. The circulation smoke discharging is performed by circulating, through the circulation pump 13, gas (in this example, carbon dioxide) fed into the body cavity by the pneumoperitoneum apparatus 31 and filtrating, through a filter 16 to be described later, smoke and mist (hereinafter collectively referred to as “smoke”) generated in treatment with the electrocautery scalpel 22.

The suction port 12a is connected with the suction trocar T1 through a circulation suction tube B1.

The filter 16 is attached to the suction port 12a. The filter 16 is made of, for example, non-woven fabric and configured to filtrate sucked smoke.

The air feeding port 12b is connected with the air feeding trocar T3 through a circulation air feeding tube B2.

The circulation pump 13 is connected with the circulation flow rate control unit 15 and driven based on a control signal from the circulation flow rate control unit 15 to suck air through the circulation suction tube B1 connected with the suction port 12a and feed air through the circulation air feeding tube B2 connected with the air feeding port 12b. Namely, the circulation pump 13 is a pump for the circulation smoke discharging in the circulation smoke discharging apparatus 11.

The connection units 14a and 14b are each a connector connectable with a communication cable. The connection unit 14a is connected with the treatment instrument apparatus 21 through a communication cable C1. The connection unit 14b is connected with the pneumoperitoneum apparatus 31 through a communication cable C2.

The circulation flow rate control unit 15 is connected with the connection units 14a and 14b and the circulation pump 13. The circulation flow rate control unit 15 includes a central processing unit (hereinafter referred to as a “CPU”) 17, and a memory 18 including a ROM and a RAM. Functions of the circulation flow rate control unit 15 are achieved by the CPU 17 executing various computer programs stored in the memory 18.

The circulation flow rate control unit 15 is configured to control a circulation flow rate by outputting a pump input voltage to be described later to the circulation pump 13. More specifically, the circulation flow rate control unit 15 controls the circulation flow rate by acquiring the setting output information from the treatment instrument apparatus 21 through the communication cable C1 connected with the connection unit 14a, determining, through an input voltage determination unit 18a, a circulation pump input voltage in accordance with the setting output information, converting voltage supplied from an external power source (not illustrated) into the circulation pump input voltage, and outputting the circulation pump input voltage to the circulation pump 13.

The circulation flow rate control unit 15 is configured to start or stop the circulation smoke discharging in response to a control signal inputted from the pneumoperitoneum apparatus 31 through the communication cable C2 connected with the connection unit 14b.

The memory 18 stores various computer programs including the input voltage determination unit 18a.

The input voltage determination unit 18a is a processing unit configured to determine the pump input voltage to be a voltage corresponding to the circulation flow rate in accordance with the setting output information of the treatment instrument apparatus 21 based on the setting output information of the treatment instrument apparatus 21.

More specifically, the input voltage determination unit 18a determines the pump input voltage corresponding to a predetermined circulation flow rate Z when the electrocautery scalpel is in an off state.

The input voltage determination unit 18a also determines the pump input voltage corresponding to the circulation flow rate in accordance with an output level of the electrocautery scalpel 22.

The input voltage determination unit 18a determines the pump input voltage so that the circulation flow rate becomes equal to a first circulation flow rate when a set mode of the treatment instrument is a dissection mode as a first mode. The input voltage determination unit 18a determines the pump input voltage so that the circulation flow rate becomes equal to a second circulation flow rate smaller than the first circulation flow rate when the set mode of the treatment instrument is a coagulation mode as a second mode.

As illustrated in FIG. 1, the treatment instrument apparatus 21 includes an electrocautery scalpel apparatus body 23, the electrocautery scalpel 22 as the treatment instrument, and a foot switch 24. The treatment instrument apparatus 21 is configured to output the setting output information of the electrocautery scalpel 22 to the circulation smoke discharging apparatus 11.

The electrocautery scalpel apparatus body 23 is configured to output high frequency current to the electrocautery scalpel 22. The set mode of the electrocautery scalpel apparatus body 23 is configured to be switched in accordance with an instruction inputted to an operation section 22a to be described later or the foot switch 24 by a surgeon. The electrocautery scalpel apparatus body 23 outputs the high frequency current in accordance with the set mode to the electrocautery scalpel 22. The set mode includes the dissection mode and the coagulation mode.

An output level of the electrocautery scalpel apparatus body 23 is adjustable to, for example, “High”, “Middle”, or “Low” in accordance with an instruction inputted to an operation section (not illustrated) by the surgeon.

The electrocautery scalpel apparatus body 23 outputs the setting output information of the high frequency current outputted to the electrocautery scalpel 22, to the circulation flow rate control unit 15 of the circulation smoke discharging apparatus 11 through the communication cable C1. The setting output information includes set mode information of the electrocautery scalpel 22 and output level information of the electrocautery scalpel 22.

The dissection mode is a mode in which the high frequency current is outputted to an affected part tissue to dissect the affected part tissue through vaporization with Joule heat. The coagulation mode is a mode in which the high frequency current is intermittently outputted to the affected part tissue to thermally coagulate the affected part tissue with Joule heat. When the surgeon sets the electrocautery scalpel 22 to the dissection mode and performs treatment on an affected part, smoke in an amount larger than the amount of smoke in treatment in the coagulation mode is generated in the body cavity, and thus the circulation smoke discharging is required in a larger amount.

The electrocautery scalpel 22 is connected with the electrocautery scalpel apparatus body 23 and inserted into the body cavity through the treatment instrument trocar T2. The electrocautery scalpel 22 outputs the high frequency current supplied from the electrocautery scalpel apparatus body 23, to the affected part from a chip (not illustrated) provided at a distal end of the electrocautery scalpel 22. The electrocautery scalpel 22 includes the operation section 22a, and the surgeon can input a mode switching instruction by operating the operation section 22a with fingers.

Note that the electrocautery scalpel 22 may be of any type such as a monopolar type, a bipolar type, a ball chip type, or a tweezers type. The chip at the distal end of the electrocautery scalpel 22 may have any shape such as a blade shape, a needle shape, a ball shape, or a loop shape.

The foot switch 24 is configured to perform inputting of an instruction such as the set mode switching instruction by a foot of the surgeon.

The pneumoperitoneum apparatus 31 is connected with the connection unit 14b of the circulation smoke discharging apparatus 11. The pneumoperitoneum apparatus 31 is connected with the air feeding trocar T3 through a pneumoperitoneum tube B3. The pneumoperitoneum apparatus 31 is connected with a tank 32 filled with gas (in this example, carbon dioxide).

The pneumoperitoneum apparatus 31 includes an air feeding valve 33, a pressure sensor 34, a relief valve 35, and a pneumoperitoneum control unit 36. The pneumoperitoneum apparatus 31 feeds air into the body cavity of the patient K until a body cavity internal pressure reaches a set pressure, thereby expanding a volume of the inside of the body cavity of the patient K so that an appropriate endoscope image can be obtained by the endoscope apparatus 41 and a space is provided for work with the electrocautery scalpel 22. The set pressure is set to be, for example, any pressure in a range of 5 mmHg to 20 mmHg in accordance with a surgical form.

The air feeding valve 33 is provided to a pneumoperitoneum pipe line 37 connected with the pneumoperitoneum tube B3. The air feeding valve 33 is connected with the pneumoperitoneum control unit 36 and configured to open and close in response to a control signal from the pneumoperitoneum control unit 36. The pneumoperitoneum control unit 36 opens the air feeding valve 33 to feed air from the tank 32 into the body cavity of the patient K through the pneumoperitoneum tube B3 connected with the air feeding trocar T3.

The pressure sensor 34 is provided to the pneumoperitoneum pipe line 37. The pressure sensor 34 is connected with the pneumoperitoneum control unit 36. The pressure sensor 34 measures the body cavity internal pressure in the body cavity by measuring pressure in the pneumoperitoneum pipe line 37, and outputs information of the measured body cavity internal pressure to the pneumoperitoneum control unit 36.

The relief valve 35 is provided to the pneumoperitoneum pipe line 37. The relief valve 35 is connected with the pneumoperitoneum control unit 36. When a control signal indicating that the body cavity internal pressure exceeds a set value is received from the pneumoperitoneum control unit 36, the relief valve 35 is opened to reduce the body cavity internal pressure.

The pneumoperitoneum control unit 36 includes a CPU 38 and a memory 39 including a ROM and a RAM. Functions of the pneumoperitoneum control unit 36 are achieved by the CPU 38 executing various computer programs stored in the memory 39.

The memory 39 stores various computer programs including a pneumoperitoneum processing computer program 39a.

The pneumoperitoneum processing computer program 39a is a processing unit configured to perform pneumoperitoneum processing. The pneumoperitoneum processing computer program 39a transmits a control signal to start the circulation smoke discharging to the circulation smoke discharging apparatus 11 when the body cavity internal pressure measured by the pressure sensor 34 reaches the set pressure. The pneumoperitoneum processing computer program 39a transmits a control signal to open the relief valve 35 to the relief valve 35 when the body cavity internal pressure exceeds the set value. The set pressure is set in advance to a pressure suitable for surgery performed by expanding the inside of the body cavity.

The endoscope apparatus 41 includes an endoscope 42, a light source apparatus 43, a processor 44, and a monitor 45.

The endoscope 42 is connected with the processor 44 and the light source apparatus 43 and inserted into the body cavity through the air feeding trocar T3. The endoscope 42 includes an observation window (not illustrated) at a distal end and is capable of performing image pickup of the inside of the body cavity.

The light source apparatus 43 is configured to supply illumination light to the endoscope 42 by guiding, through a light guiding member, light emitted from a semiconductor light source and adjusting, for example, color and light intensity distribution through an optical conversion member provided at a distal end of the light guiding member.

The processor 44 is configured to supply electrical power to the endoscope 42, process an image picked up by the endoscope 42 to generate image information, and output the image information to the monitor 45 connected with the processor 44.

The monitor 45 is configured to display the image information inputted from the processor 44.

### (Effects)

The following describes the pneumoperitoneum processing by the pneumoperitoneum apparatus 31.

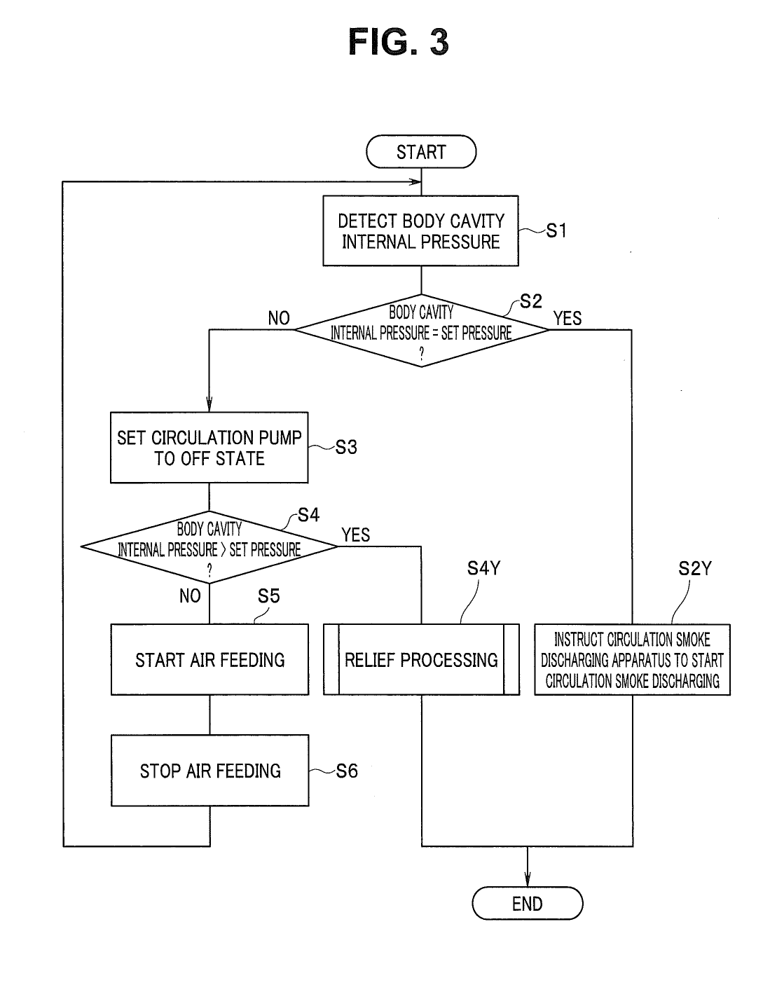

FIG. 3 is a flowchart illustrating a process of the pneumoperitoneum processing by the circulation smoke discharging system 1 according to the embodiment of the present invention.

When the surgeon inputs a processing start instruction to the pneumoperitoneum apparatus 31 through the operation section (not illustrated), the CPU 38 reads the pneumoperitoneum processing computer program 39a from the memory 39 and starts the pneumoperitoneum processing. The processing start instruction inputted to the pneumoperitoneum apparatus 31 is also inputted to the circulation smoke discharging apparatus 11 through the communication cable C2. The CPU 17 reads various computer programs related to circulation smoke discharging processing and including the input voltage determination unit 18a, and prepares for start of the circulation smoke discharging processing.

The pneumoperitoneum control unit 36 detects the body cavity internal pressure (step (hereinafter abbreviated as “S”) 1). At S1, the pneumoperitoneum control unit 36 detects the body cavity internal pressure measured by the pressure sensor 34.

The pneumoperitoneum control unit 36 determines whether the body cavity internal pressure is equal to the set pressure (S2). At S2, the pneumoperitoneum control unit 36 determines whether the body cavity internal pressure acquired by the pressure sensor 34 is equal to the set pressure, which is set in advance. When it is determined that the body cavity internal pressure is equal to the set pressure (Yes at S2), the processing proceeds to S2Y. When it is determined that the body cavity internal pressure is different from the set pressure (No at S2), the processing proceeds to S3. Note that the body cavity internal pressure and the set pressure may be determined to be equal to each other when a difference between the body cavity internal pressure and the set pressure is in a predetermined range.

At S2Y, a control signal instructing to start the circulation smoke discharging is transmitted to the circulation smoke discharging apparatus 11. Having received the control signal inputted from the pneumoperitoneum control unit 36, the circulation smoke discharging apparatus 11 starts the circulation smoke discharging processing (S21 to S24) to be described later. After having transmitted the control signal at S2Y, the pneumoperitoneum control unit 36 ends the pneumoperitoneum processing.

The pneumoperitoneum control unit 36 sets the circulation pump 13 to the off state (S3). The pneumoperitoneum control unit 36 transmits a control signal to stop operation of the circulation pump 13 to the circulation flow rate control unit 15. Having received the control signal, the circulation flow rate control unit 15 sets the circulation pump 13 to the off state and stops the operation.

After S3, the pneumoperitoneum control unit 36 determines whether the body cavity internal pressure exceeds the set pressure (S4). At S4, the pneumoperitoneum control unit 36 compares the body cavity internal pressure acquired by the pressure sensor 34 with the set pressure. When it is determined that the body cavity internal pressure does not exceed the set pressure (No at S4), the processing proceeds to S5. When it is determined that the body cavity internal pressure exceeds the set pressure (Yes at S4), the pneumoperitoneum control unit 36 proceeds to S31 to perform relief processing at S4Y.

At S5, the pneumoperitoneum control unit 36 starts air feeding. At S5, the pneumoperitoneum control unit 36 opens the air feeding valve 33 to feed gas into the body cavity of the patient K through the pneumoperitoneum tube B3 for a predetermined time period P. The predetermined time period P is, for example, one second.

After S5, the pneumoperitoneum control unit 36 stops the air feeding (S6). At S6, the pneumoperitoneum control unit 36 stops the air feeding and returns the processing to S1.

When the body cavity internal pressure is increased to the set pressure through the processing at S1 to S6 so that the body cavity internal pressure is equal to the set pressure, a control signal instructing to start the circulation smoke discharging processing is transmitted to the circulation smoke discharging apparatus 11.

The processing at Si to S6 constitutes the pneumoperitoneum processing.

The following describes the circulation smoke discharging processing by the circulation smoke discharging apparatus 11.

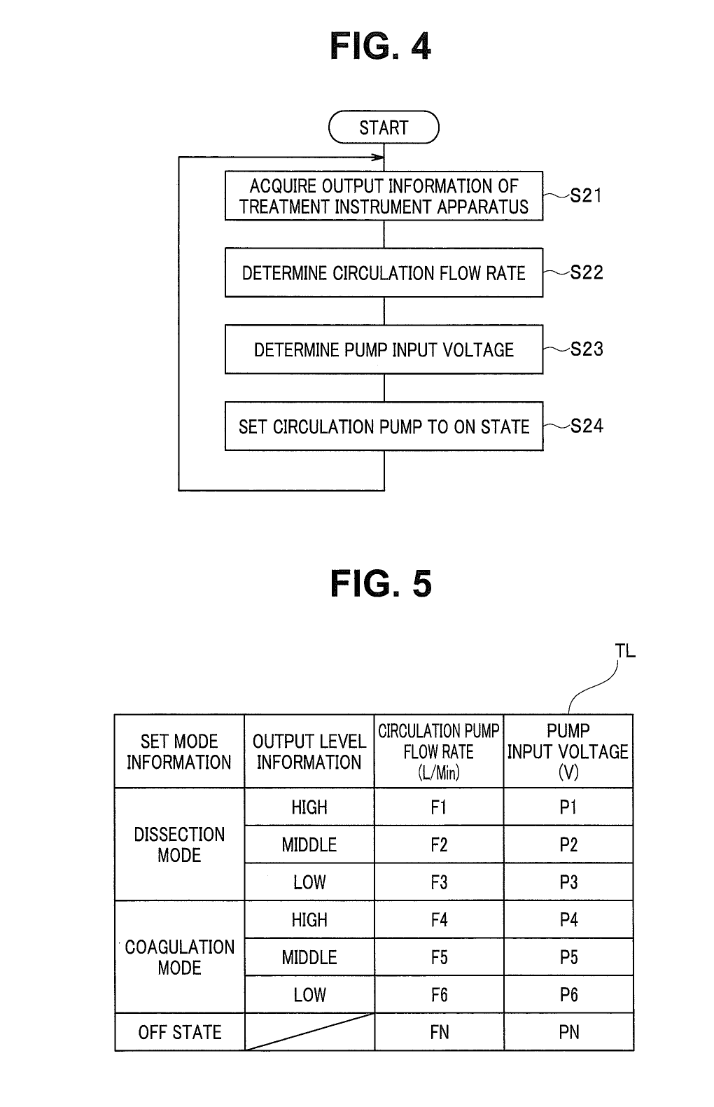

FIG. 4 is a flowchart illustrating a process of the circulation smoke discharging processing by the circulation smoke discharging apparatus 11 of the circulation smoke discharging system 1 according to the embodiment of the present invention. FIG. 5 is an explanatory diagram for description of an exemplary flow rate voltage table TL of the circulation smoke discharging system 1 according to the embodiment of the present invention.

At S2Y, when the control signal instructing to start the circulation smoke discharging is received from the pneumoperitoneum control unit 36, the circulation flow rate control unit 15 starts the circulation smoke discharging processing.

The circulation flow rate control unit 15 acquires the setting output information of the treatment instrument apparatus 21 (S21). The circulation flow rate control unit 15 acquires the setting output information of the treatment instrument apparatus 21 through the connection unit 14a.

The circulation flow rate control unit 15 determines the circulation flow rate (S22). At S22, the circulation flow rate control unit 15 starts processing of the input voltage determination unit 18a and determines, through the processing of the input voltage determination unit 18a, the circulation flow rate in accordance with the setting output information acquired from the treatment instrument apparatus 21 by referring to the flow rate voltage table TL.

FIG. 5 illustrates the exemplary flow rate voltage table TL. In FIG. 5, a flow rate of the circulation pump 13 is determined based on the set mode information and the output level information included in the setting output information. For example, the flow rate of the circulation pump 13 is determined to be F1 L/min (F1 liters per minute) when the electrocautery scalpel 22 is in the dissection mode and the output level information is “High”; the flow rate of the circulation pump 13 is determined to be F2 L/min when the electrocautery scalpel 22 is in the dissection mode and the output level information is “Middle”; and the flow rate of the circulation pump 13 is determined to be F3 L/min when the electrocautery scalpel 22 is in the dissection mode and the output level information is “Low”. The flow rate of the circulation pump 13 is determined to be F4 L/min when the electrocautery scalpel 22 is in the coagulation mode and the output level information is “High”; the flow rate of the circulation pump 13 is determined to be F5 L/min when the electrocautery scalpel 22 is in the coagulation mode and the output level information is “Middle”; and the flow rate of the circulation pump 13 is determined to be F6 L/min when the electrocautery scalpel 22 is in the coagulation mode and the output level information is “Low”. The flow rate of the circulation pump 13 is determined to be FN L/min when the electrocautery scalpel 22 is in the off state.

The circulation flow rate control unit 15 determines the pump input voltage (S23). At S23, the circulation flow rate control unit 15 determines, through the processing of the input voltage determination unit 18a, the pump input voltage based on the flow rate of the circulation pump 13 determined at S22 by referring to the flow rate voltage table TL.

For example, in FIG. 5, at S22, the pump input voltage is determined to be P1 V (volt) when the flow rate of the circulation pump 13 is determined to be F1 L/min. Similarly, the pump input voltage is determined to be P2V, P3V, P4V, P5V, P6V, or PNV when the flow rate of the circulation pump 13 is determined to be F2 L/min, F3 L/min, F4 L/min, F5 L/min, F6 L/min, or FN L/min, respectively.

Note that the flow rate voltage table TL illustrated in FIG. 5 is merely exemplary, and the present invention is not limited to the table illustrated in FIG. 5.

The circulation flow rate control unit 15 sets the circulation pump 13 to an on state (S24). At S24, the circulation flow rate control unit 15 sets the circulation pump 13 to the on state and performs the circulation smoke discharging based on the pump input voltage determined at S23. After S24, the processing returns to S21. Accordingly, the circulation smoke discharging is performed at the circulation flow rate in accordance with the setting output information of the treatment instrument apparatus 21, for example, when the electrocautery scalpel 22 is switched from the dissection mode to the coagulation mode and the output level information of the electrocautery scalpel 22 is changed, or when the electrocautery scalpel 22 is switched from the on state to the off state.

The processing at S22 and S23 constitutes the processing of the input voltage determination unit 18a.

The following describes the relief processing by the pneumoperitoneum apparatus 31.

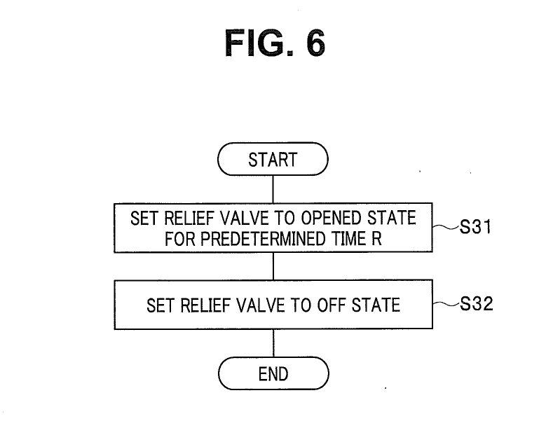

FIG. 6 is a flowchart illustrating a process of the relief processing by the circulation smoke discharging system 1 according to the embodiment of the present invention.

The pneumoperitoneum control unit 36 opens the relief valve 35 for a predetermined time period R (S31). At S31, the pneumoperitoneum control unit 36 transmits, to the relief valve 35, a control signal to open the relief valve 35 for the predetermined time period R. Having received the control signal from the pneumoperitoneum control unit 36, the relief valve 35 is opened for the predetermined time period R to externally discharge gas in the body cavity.

After S31, the pneumoperitoneum control unit 36 closes the relief valve 35 (S32). After the processing at S32, the pneumoperitoneum control unit 36 ends the pneumoperitoneum processing and the relief processing.

The processing at S31 and S32 constitutes the relief processing.

According to the above-described embodiment, the circulation smoke discharging is performed in accordance with the set mode and the output level of the treatment instrument apparatus 21, and the circulation smoke discharging can be performed in a necessary amount when necessary.

### Modifications of Embodiment

In the above-described embodiment, air from the circulation pump 13 is directly fed to the air feeding port 12b. However, in a modification of the embodiment, a flow rate adjustment valve 19 (illustrated with dashed and double-dotted lines in FIG. 2) may be provided between the circulation pump 13 and the air feeding port 12b.

The flow rate adjustment valve 19 is connected with the circulation flow rate control unit 15 and configured to open and close in response to a control signal from the circulation flow rate control unit 15.

With this configuration, the flow rate in the circulation smoke discharging can be more accurately adjusted, and the circulation smoke discharging can be performed in a necessary amount when necessary.

Note that, in the embodiment, the circulation flow rate is changed promptly when the electrocautery scalpel 22 is changed from the on state to the off state. However, the circulation flow rate may be set to the predetermined circulation flow rate Z when a predetermined time period D elapses after the electrocautery scalpel 22 is set to the off state. The predetermined time period D is, for example, any time period in a range of one second to 10 seconds. In addition, note that a plurality of predetermined time periods D may be set in accordance with the set mode and the output level of the electrocautery scalpel 22. With this configuration, the circulation smoke discharging continues for the predetermined time period D after the electrocautery scalpel 22 is set to the off state, thereby more reliably sucking and removing smoke.

Note that, in the embodiment, the circulation smoke discharging is started when the body cavity internal pressure reaches the set pressure. However, the electrocautery scalpel 22 may be set to the on state after an instruction to set the electrocautery scalpel 22 to the on state is inputted through the operation section (not illustrated) and the circulation smoke discharging apparatus 11 starts the circulation smoke discharging. More specifically, the treatment instrument apparatus 21 may start the circulation smoke discharging by transmitting a control signal to the circulation smoke discharging apparatus 11 (as illustrated with a dashed line arrow C1a in FIG. 1) after the instruction is inputted through the operation section. Then, the circulation smoke discharging apparatus 11 may transmit a control signal to the treatment instrument apparatus 21 after the circulation smoke discharging apparatus 11 starts the circulation smoke discharging (as illustrated with a dashed line arrow C1b in FIG. 1). After having received the control signal from the circulation smoke discharging apparatus 11, the treatment instrument apparatus 21 may set the electrocautery scalpel 22 to the on state. With this configuration, the circulation smoke discharging is already started when the electrocautery scalpel 22 is set to the on state, thereby sucking and removing smoke generated at a moment when the electrocautery scalpel 22 switches to the on state.

Note that, in the embodiment, the set mode of the electrocautery scalpel 22 includes the dissection mode and the coagulation mode, but the present invention is not limited to the set modes. The set mode of the electrocautery scalpel 22 may include any other mode such as a spray coagulation mode for stopping bleeding from a wide range of tissue by arc discharge.

Note that, in the embodiment, the input voltage determination unit 18a determines the circulation pump input voltage based on the flow rate voltage table. However, the flow rate voltage table does not necessarily need to be provided, and the circulation pump input voltage may be determined by using any predetermined formula.

Note that, in the embodiment, the input voltage determination unit 18a is disposed in the circulation smoke discharging apparatus 11, but may be disposed in the pneumoperitoneum apparatus 31. In this case, the setting output information of the treatment instrument apparatus 21 may be outputted to the pneumoperitoneum apparatus 31 through the circulation smoke discharging apparatus 11. Alternatively, the treatment instrument apparatus 21 and the pneumoperitoneum apparatus 31 may be connected with each other through a communication cable (not illustrated) so that the setting output information is directly outputted from the treatment instrument apparatus 21 to the pneumoperitoneum apparatus 31.

The present invention is not limited to the above-described embodiment but may be provided with, for example, various kinds of changes and modifications without departing from the scope of the present invention.

The circulation smoke discharging system 1 according to the embodiment removes smoke generated in treatment with the electrocautery scalpel 22 by sucking the smoke through the circulation smoke discharging apparatus 11. However, smoke generated in treatment with the electrocautery scalpel 22 may be removed by feeding air through an air feeding pipe line of the endoscope.

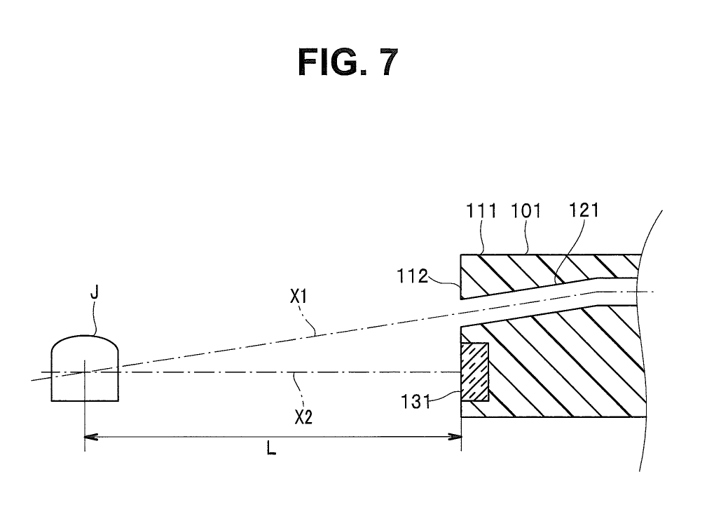

FIG. 7 is an explanatory cross-sectional view for description of a configuration of an endoscope insertion section distal end 111 of an endoscope 101.

The endoscope insertion section distal end 111 includes an objective lens 131 and an air feeding pipe line 121.

The objective lens 131 is provided to an endoscope insertion section distal-end surface 112 and configured to take in reflected light from a treatment site J. An image pickup device (not illustrated) is provided behind the objective lens 131 and configured to convert the reflected light from the treatment site J into image information.

The air feeding pipe line 121 is provided in the endoscope insertion section distal end 111 and connected with an endoscope body (not illustrated) to feed air toward the endoscope insertion section distal-end surface 112 through the endoscope body. Note that the air feeding pipe line 121 may be included in the endoscope insertion section distal end 111 or may be externally provided along an outer periphery of the endoscope insertion section distal end 111.

The air feeding pipe line 121 has a central axis X1 tilted toward an optical axis X2 of the objective lens 131 so that gas fed out of the air feeding pipe line 121 collides with smoke generated from the treatment site J. FIG. 7 illustrates an example in which the treatment site J is positioned at a position at which the optical axis X2 of the objective lens 131 intersects with the central axis X1 of the air feeding pipe line 121 and that is separated from the objective lens 131 by a predetermined distance L (the distance L is, for example, a focal length of the objective lens 131). The predetermined distance L is, for example, 100 mm.

According to the endoscope 101 illustrated in FIG. 7, air can be fed to the treatment site J, thereby efficiently blowing off any floating substance in an observation visual field of the endoscope 101 to obtain a favorable view.

Note that it may be automatically sensed through image processing by a floating substance sensing unit (not illustrated) that smoke generated from the treatment site J floats or falls into the observation visual field of the endoscope 101, and then the smoke may be blown off with gas fed from the air feeding pipe line 121. When any floating substance is sensed by the floating substance sensing unit, air may be fed through the air feeding pipe line 121 while air in an amount same as an amount of the fed air may be taken in by an air intake unit (not illustrated). The air feeding and the air intake may be stopped when no floating substance is sensed any more. In this manner, the air feeding can be performed without changing the body cavity internal pressure.

With this configuration, any floating substance can be more reliably sucked by starting an air feeding operation after start of a suction operation to optimally control start timings of the operations.

The present invention can provide a circulation smoke discharging system capable of performing circulation smoke discharging in a necessary amount when necessary.

What is claimed is:

1. A circulation smoke discharging system comprising:
a reception unit configured to receive setting output information of a treatment instrument apparatus including a treatment instrument;
a circulation flow rate information setting unit storing a plurality of pieces of the setting output information and a plurality of circulation flow rates associated with the plurality of pieces of the setting output information, and configured to set one of the plurality of circulation flow rates in accordance with the setting output information;
a circulation smoke discharging apparatus configured to feed gas at the circulation flow rate set by the circulation flow rate information setting unit in accordance with a result of the reception by the reception unit and pedal' circulation smoke discharging by sucking smoke generated in a body cavity and gas in the body cavity;
a pump for the circulation smoke discharging;
an input voltage determination unit configured to determine a pump input voltage corresponding to the circulation flow rate; and
a circulation flow rate control unit configured to control the circulation flow rate by outputting the pump input voltage to the pump.

2. The circulation smoke discharging system according to claim 1, wherein, when the treatment instrument is in an off state, the input voltage determination unit determines the pump input voltage to be a voltage corresponding to the predetermined circulation flow rate.

3. The circulation smoke discharging system according to claim 1, wherein the input voltage determination unit:
determines the pump input voltage so that the circulation flow rate becomes equal to a first circulation flow rate when a set mode of the treatment instrument is a first mode; and
determines the pump input voltage so that the circulation flow rate becomes equal to a second circulation flow rate when the set mode of the treatment instrument is a second mode.

4. The circulation smoke discharging system according to claim 3, wherein
the first mode is a dissection mode,
the second mode is a coagulation mode, and
the second circulation flow rate is smaller than the first circulation flow rate.

5. The circulation smoke discharging system according to claim 1, wherein the input voltage determination unit determines the pump input voltage corresponding to the circulation flow rate in accordance with an output level of the treatment instrument.

6. The circulation smoke discharging system according to claim 1, wherein the circulation flow rate control unit sets the circulation flow rate to be a predetermined circulation flow rate when a predetermined time period elapses after the treatment instrument is set to an off state.

7. The circulation smoke discharging system according to claim 1, further comprising a connection unit connected with a cable through which the setting output information is received, wherein the circulation smoke discharging apparatus is capable of inputting the setting output information of the treatment instrument through the connection unit.

8. The circulation smoke discharging system according to claim 1, wherein the treatment instrument of the treatment instrument apparatus is set to an on state after an operation instruction is performed and the circulation smoke discharging apparatus starts the circulation smoke discharging.

9. The circulation smoke discharging system according to claim 1, further comprising a pneumoperitoneum apparatus connected with the circulation smoke discharging apparatus, wherein the circulation smoke discharging apparatus starts or stops the circulation smoke discharging in response to a control signal inputted from the pneumoperitoneum apparatus.

10. The circulation smoke discharging system according to claim 1, wherein
the pump and the circulation flow rate control unit are disposed in the circulation smoke discharging apparatus, and
the circulation flow rate control unit includes the input voltage determination unit.

11. The circulation smoke discharging system according to claim 1, wherein the treatment instrument is an electrocautery scalpel.

12. The circulation smoke discharging system according to claim 1, wherein the setting output information is at least one of set mode information, output level information, a flow rate of the pump, and the pump input voltage.

13. The circulation smoke discharging system according to claim 1, wherein the circulation flow rate control unit performs control so that gas circulation does not stop but gas circulates at the predetermined circulation flow rate when the setting output information of the treatment instrument is less than a predetermined value.
